(19) United States
(12) Patent Application Publication
(10) Pub. No.: US 2006/0111942 A1
(43) Pub. Date: May 25, 2006

(54) Consultation service, system, and method for selecting an electronic medical record (EMR) provider

(75) Inventors: Eric S. Fishman, Palm Beach Gardens, FL (US); Ann D. Fishman, Palm Beach Gardens, FL (US); Gregory S. Skillman, North Palm Beach, FL (US); Krish Michaels, Boynton Beach, FL (US); Brent Holmes, Palm Beach Gardens, FL (US); Jimmy Pedroso, Wellington, FL (US)

Correspondence Address: AKERMAN SENTERFITT, P.O. BOX 3188, WEST PALM BEACH, FL, 33402-3188, US

(73) Assignee: 1450, Inc., Palm Beach Gardens, FL (US)

(21) Appl. No.: 10/955,262

(22) Filed: Sep. 30, 2004

(51) Int. Cl. A61B 5/00; G06F 19/00

(52) U.S. Cl. 705/3

(57) ABSTRACT

A system for searching for vendor products including a consultant data store, a search engine, and a client interface having an input section and a result section. The consultant data store can be populated with standardized product characteristics for a plurality of products supplied by different vendors. The search engine can compare customer preferences against product characteristic contained in the consultant data store. The input section can be configured for customers to enter customer preferences into a plurality of consultant established fields. The result section can be configured to present a plurality of vendor products based upon search engine results, wherein an indicator based upon a level of correspondence between customer preferences and product characteristics is displayed next to each vendor product. In one embodiment, the products can be electronic medical record products and the client interface can be a Web-based interface configured for health care providers.

### BACKGROUND

1. Field of the Invention

The present invention relates to the field of information technology and, more particularly, to a consultation service for matching health care providers with an electronic medical record solution.

2. Description of the Related Art

While the majority of health care providers (HCP) and hospitals utilize advanced medical diagnosis equipment, HCP records are typically maintained in hardcopy as opposed to being maintained in an electronic form. Hardcopy record maintenance for medicine can be attributed to traditional business practices, a vast array of disparate records types (such as x-rays, physician notes, medical charts, laboratory results, and the like), patient privacy concerns, HCP liability concerns, and a general lack of electronic information processing experience within the medical community.

Hardcopy maintenance of medical records is a very costly endeavor to HCPs and patients. HCPs typically require significant warehousing for the physical records, which must be maintained for an extended period. Additionally, a large support staff is necessary to properly retrieve, store, copy, and update hardcopy medical records. Also a reliance upon hardcopy medical records can result in lost records, extensive appointment delays attributable to record access times, patient-doctor lock-in, and a lack of crucial patient information in emergency care situations. Government studies have estimated that a large scale conversion of medical records from hardcopy to an electronic form could result in a ten percent or more cost reduction for heath care without reducing physician salaries.

HCPs desiring to move from hardcopy record storage to electronic technologies can face a substantial challenge in locating an appropriate electronic medical record solution. HCPs are often daunted by the task of selecting an electronic medical record (EMR) product from the hundreds that exist. HCPs are often swayed by clever sales pitches, demonstrations, or cost when deciding upon an EMR product. Ill selected EMR solutions, however, can result in an EMR product that is overly cumbersome, overly expensive, or insufficiently fulfills the needs of the HCP. What is needed is a simple, cost-effective means of assisting HCP in searching for an appropriate EMR product.

### SUMMARY OF THE INVENTION

The present invention provides a system, method, and apparatus for assisting a health care provider (HCP) in finding an electronic medical record (EMR) product appropriate for the specific needs of the HCP. More specifically, a database can be constructed that includes standardized characteristics for a multitude of EMR products. An independent consultant can validate database entries for each EMR product to ensure accuracy. a comparison of characteristics of the HCP providers contained in the favored list. The contact mechanism can facilitate communications between the HCP and at least one EMR producer having an EMR product selected from the favored list.

Another aspect of the present invention can include a service for matching EMR products to HCPs. The service can include the step of registering a multitude of EMR products with the service. The registering step can include receiving and validating EMR product specific values for a multitude of EMR standardized characteristics. One or more HCPs can subscribe to the service. Subscribing can include receiving HCP specific values for core characteristics, which are a subset of the standardized characteristics. A culled list of EMR products can be generated for subscribed HCPs based on a correspondence between the EMR specific values and the HCP specific values for the core characteristics. HCP specific values can also be received for supplemental characteristics, which are another subset of the standardized characteristics. A favored list of EMR products can be constructed based upon a correspondence between the EMR specific values and the HCP specific values for the supplemental characteristics. The favored list can be presented to the HCP.

Yet another aspect of the present invention can include a system for searching for vendor products. The system can include a consultant data store, a search engine, and a client interface having an input section and a result section. The consultant data store can be populated with standardized product characteristics for a plurality of products supplied by different vendors. The search engine can compare customer preferences against product characteristic contained in the consultant data store. The input section can be configured for customers to enter customer preferences into a plurality of consultant established fields. The result section can be configured to present a plurality of vendor products based upon search engine results, wherein an indicator based upon a level of correspondence between customer preferences and product characteristics is displayed next to each vendor product. In one embodiment, the products can be electronic medical record products and the client interface can be a Web-based interface configured for HCPs.

It should be noted that the invention can be implemented as a program for controlling a computer to implement the functions described herein, or a program for enabling a computer to perform the process corresponding to the steps disclosed herein. This program may be provided by storing the program in a magnetic disk, an optical disk, a semiconductor memory, any other recording medium, or distributed via a network.

### BRIEF DESCRIPTION OF THE DRAWINGS

There are shown in the drawings, embodiments which are presently preferred, it being understood, however, that the invention is not limited to the precise arrangements and instrumentalities shown.

FIG. 1 is a schematic diagram for a system supporting health care providers (HCPs) in the selection of an electronic medical record (EMR) solution in accordance with an embodiment of the present invention.

FIG. 2 illustrates a client GUI in accordance with an embodiment of the present invention.

FIG. 3 illustrates another client GUI in accordance with an embodiment of the present invention.

FIG. 4 is a GUI that can be used by an agent of an EMR consultation service in accordance with an embodiment of the inventive arrangements presented herein.

FIG. 5 is a GUI that can be used by an EMR producer to interact with a consultation service in accordance with an embodiment of the inventive arrangements presented herein.

FIG. 6 is a flow chart illustrating a method for matching EMR products to HCPs in accordance with an embodiment of the inventive arrangements disclosed herein.

### DETAILED DESCRIPTION OF THE INVENTION

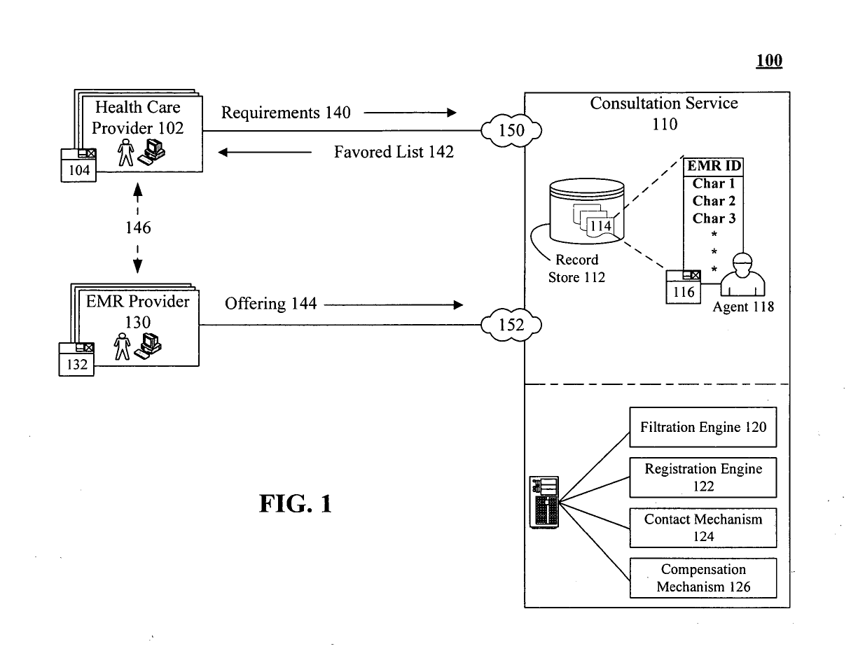

FIG. 1 is a schematic diagram for a system 100 supporting health care providers (HCPs) in the selection of an electronic medical record (EMR) solution. The system 100 can include consultation service 110 that assists HCP 102 in selecting a suitable EMR solution from offerings of multiple EMR producers 130.

In system 100, each EMR producer 130 can use EMR interface 132 to convey product offerings 144 to the consultation service 110 via network 152. Data from the offerings 144 can be recorded in records 114 within data store 112. Each EMR producer 130 can be assigned a unique key or EMR ID used by the record store. The records 114 associated with products of one or more EMR producers 130 can include multiple standardized characteristics that are associated with a specific EMR product. The characteristics can include a series of core characteristics common to all EMR products, as well as additional supplemental characteristics, which can be unique to a subset of EMR products.

Core characteristics can include, but are not limited to, product cost, operating system, supported HCP size, Web-based or local functionality, interconnectivity options, types of supported records (graphics, video, X-Ray, sound), security, guaranteed uptime, live and onsite product support, and the like. Supplemental characteristics can include, but are not limited to, access to medical expert systems, transcription services, automated patient billing, accounting services, interconnectivity to other medical systems, online appointment scheduling, and the like.

The consultation services 110 can independently validate the characteristics provided by the EMR producer 130 to ensure accuracy and completeness. For example, an agent 118 can evaluate EMR producer 130 products and use consultant interface 116 to modify the records 114 as needed.

One or more HCP 102 can utilize HCP interface 104 to submit requirements 140 detailing the EMR needs and desires of the HCP 102 to the consultation service 110 via network 150. The consultation service 110 can match these requirements against capabilities of EMR products previously stored within record store 112.

This match can result in a culled list of available EMR products that potentially satisfy the requirements 140. The culled list can be presented to agent 118 through consultant interface 116. The agent 118 can use the culled list to focus upon the EMR products which should be further investigated for the HCP 102. The agent 118 can further narrow the list into a small number of optimal EMR solutions tailored for the HCP 102. This narrow list, called a favored list 142 can, for example, include a top three, five, or N number of EMR products. The agent 118 can annotate each of the EMR products on the list to indicate strengths and weakness of one EMR product compared to another EMR product.

The HCP 102 can receive the favored list 142 and determine whether one of the EMR products appears satisfactory or whether the HCP 102 needs to further refine the requirements 140 to receive a new and different favored list 142. The agent 118 is available to provide live assistance to guide the HCP 102 through this potentially iterative process. The HCP 102 can also desire contact with particular EMR producers 130 having products on the favored list 142, which the consultation service 110 can facilitate.

In matching records and performing other consultation tasks, the consultation service 110 can utilize one or more of a filtration engine 120, a registration engine 122, a contact mechanism 124, and a compensation mechanism 126.

The filtration engine 120 can generate a culled list of EMR products based upon a correspondence level of the offerings 144 to the requirements 140. The culled list can be manipulated by agent 118, by HCP 102, and by imposing additional supplemental criteria specified by the HCP 102. The manipulated culled list can be processed by the filtration engine 120 to generate the favored list 142. In one embodiment, the filtration engine 120 can also include a feedback/learning mechanism (not shown).

The registration engine 122 can be used to register EMR products with the consultation service 110. The contact mechanism 124 can establish one or more channels of communication between the HCP 102 and the EMR producer 130. Using the consultation service 110 as a communication intermediary can allow the HCP 102 to receive answers to targeted questions without fear of aggressive and undesired solicitations from the EMR producer 130. The compensation mechanism 126 can be used to track interactions between HCPs 102 and EMR producers 130 so that the consultation service 110 can be assured to appropriate remuneration.

It should be appreciated that information for a sufficient number of EMR products, core characteristics, and supplemental characteristics can be a condition prerequisite to establishing a desired level of service. For example, a minimum of fifty different registered EMR products can be a lower threshold necessary for providing HCP 102 choice and consultation service 110 independence. Additionally, at least ten core characteristics can be necessary to reasonably differentiate among EMR producer 130 offerings. Further, when system 100 offers twenty or more supplemental characteristics, EMR producers 130 can minimally define their products so that potential customers can make informed product selections.

As illustrated herein, HCP interface 102, EMR interface 132, and consultant interface 116 can represent an interface into a locally implemented computer application, an interface into a remotely located Web-based application, or a combination thereof.

Networks 150 and 152 can represent any communication mechanism capable of conveying digitally encoded information. Each of the networks 150 and 152 can include a computer network like a local area network or a wide area network, a telephony network like a public switched telephone network (PSTN) or a mobile telephone network, a cable network, a satellite network, a broadcast network, and the like. Further, each of the networks 150 and 152 can use wireless as well as line-based communication pathways. Digitally encoded information can be conveyed via network 150 and 152 in accordance with any communication protocol, such as a packet-based communication protocol or a circuit based communication protocol.

Record store 112 can store information in any recording medium, such as a magnetic disk, an optical disk, a semiconductor memory, and the like. Further, record store 112 can utilize any information retention technique including a file-based storage technique or a database storage technique. Moreover, record store 112 can be a storage area fixed to a geographical location or a storage area distributed across a network space.

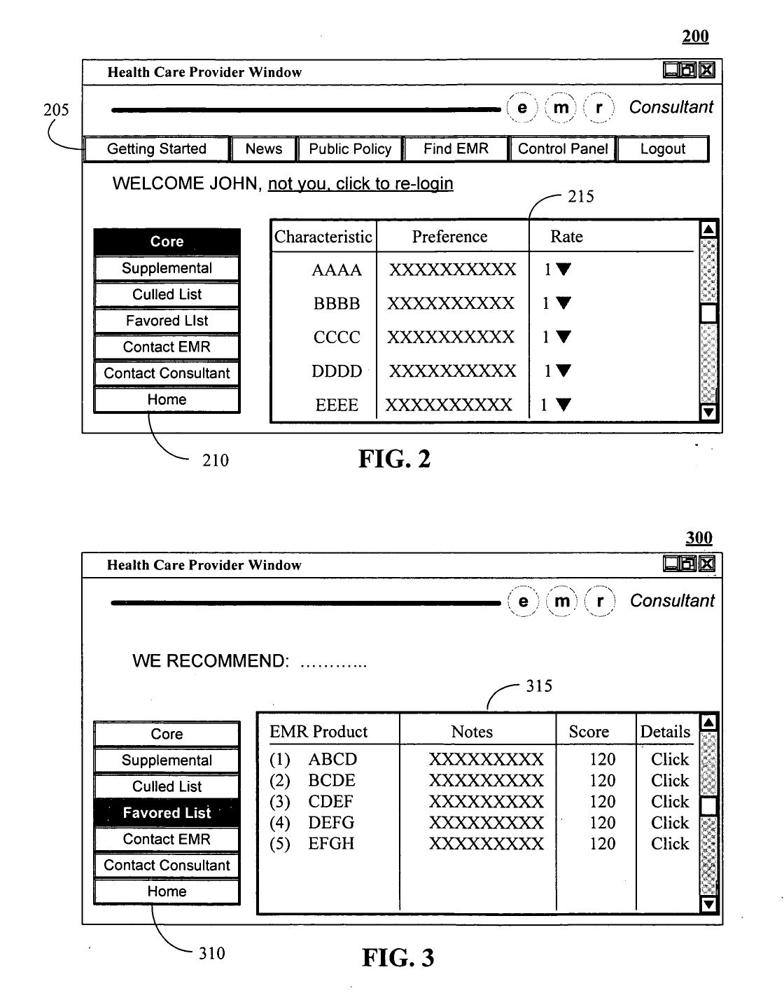

FIG. 2 and FIG. 3 can represent different stages of a client interface used to permit a client to search for vendor products. In one embodiment, the graphical user interface (GUI) 200 and 300 can represent an implementation of the HCP interface 102 of FIG. 1. It should be appreciated, however, that the invention is not limited in this regard and that the client interface illustrated in FIG. 2 and FIG. 3 can be used for search for any vendor product.

FIG. 2 illustrates GUI 200 that includes a navigation bar 205, a tab section 210, and an input section 215. The navigation bar 205 can allow a user to explore sections of the client interface that provide general information regarding EMR records. The navigation bar 205 can include buttons for “getting started”, “news”, “public policy”, “find EMR”, “control panel”, and “logout.” Getting started provides textual help for new users. News provides information pertaining to published EMR record articles. Public policy details advantages of utilizing EMR instead of hardcopy medical records. Control panel jumps the user to user configurable options.

The tab section 210 provides a quick means for the user to alter content of the GUI 200. The tab section 210 can include sections for “core”, “supplemental”, “culled list”, “favored list”, “contact EMR”, “contact consultant”, and “home”. Core can permit the entry of customer preferences for consultant established fields relating to core characteristics of EMR products. Supplemental can permit entry of customer preferences for consultant established fields relating to supplemental characteristics of EMR products. Culled list presents a list of EMR products that satisfy the core criteria provided by the user. Favored list provides a list of top EMR solutions as determined by customer criteria and consultant input. Contact EMR establishes a communication between the user and a producer of a currently selected EMR product. Contact consultant establishes a communication between the user and a consultant.

The input section 215 permits the user to enter customer preferences into the character fields. The characteristics column can represent an identifier for consultant established fields relating to core characteristics of an associated EMR product. The preference column can permit a user to enter a desired value for each characteristic. The rate column can represent a user designated criticality level for the corresponding characteristic. The rate can be to prefer one characteristic over another in accordance with user preference.

FIG. 3 illustrates GUI 300 that includes tab section 310, and a result section 315, where the tab section 310 is identical to tab section 210 of FIG. 2. Result section 315 presents a top N number of EMR products specifically selected from the needs of a HCP. The result section 315 can include columns for “EMR product”, “Notes”, “Score”, and “Details”.

The EMR product column can include a ranked list of preferred providers. The notes column can include notes added by a consultant concerning the associated EMR product. Clicking a button included in the details column can result in a window being displayed that includes further information about the selected EMR product.

The score column can present a correspondence score between the customer input preferences and the product characteristics. In one embodiment, different levels can be designed instead of a numerical value. Further, the score column can be color coded depending upon previously established correspondence levels. A user option (not shown) can permit a user to display a correspondence between provider characteristics and user entered preferences for each characteristic used to generate the correspondence score. Moreover, each displayed characteristic can be color coded to indicate a correspondence level.

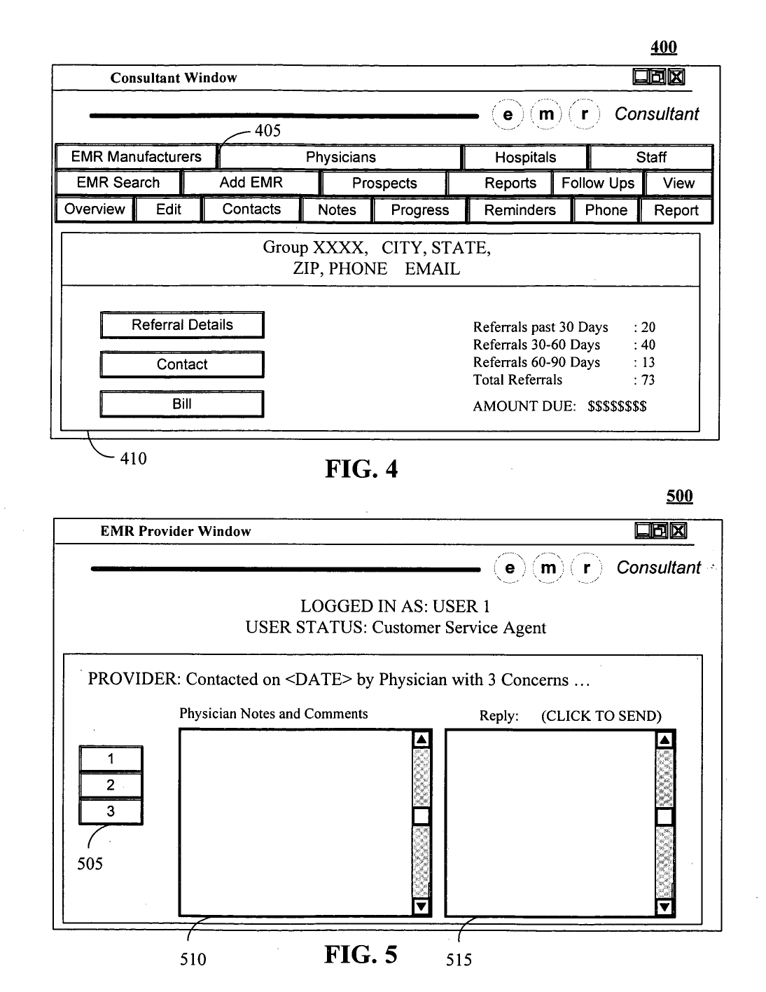

FIG. 4 is a GUI 400 that can be used by an agent of an EMR consultation service in accordance with an embodiment of the inventive arrangements presented herein. The GUI 400 can represent an implementation of the consultant interface 116 of FIG. 1.

The GUI 400 can include a navigation bar 405, and a display window 410. The navigation bar 405 can permit a consultant to navigate from one screen to another within a consultant information system. The navigation bar 405 can include such elements as “EMR Manufacturers”, “Physicians”, “Hospitals”, “Staff”, “EMR Search”, “Add EMR”, “Prospects”, “Reports”, “Follow ups”, “View”, “Overview”, “Edit”, “Contacts”, “Notes”, “Progress”, “Reminders”, “Phone”, and “Report”.

The display window 410 can present information pertinent to the tab selected in the navigation bar 405. The illustrated display window 410 includes overview information concerning referrals presented to a particular EMR product.

FIG. 5 is a GUI 500 that can be used by an EMR producer to interact with a consultation service in accordance with an embodiment of the inventive arrangements presented herein. The GUI 500 can represent an implementation of the EMR interface 132 of FIG. 1.

GUI 500 illustrates a contact screen for establishing a communication between an EMR producer and a HCP. GUI 500 includes a comment navigation bar 505, a note section 510, and a reply section 515. The comment navigation bar 505 can provide a selectable listing of comments conveyed between an EMR producer and a HCP. For each selected comment, a physician or other HCP can provide a question, comment or concern. The EMR producer can respond to each note by inputting a response in the reply section 515.

It should be noted that the GUIs of FIGS. 2-5 are for illustrative purposes only and are not be construed as an attempt to exhaustively specify all contemplated GUI formats. Consequently, the present invention is not to be limited to the precise GUI arrangements presented herein, but should be considered to include GUI derivatives within the scope of the detailed description and within the expertise of an ordinary software designer.

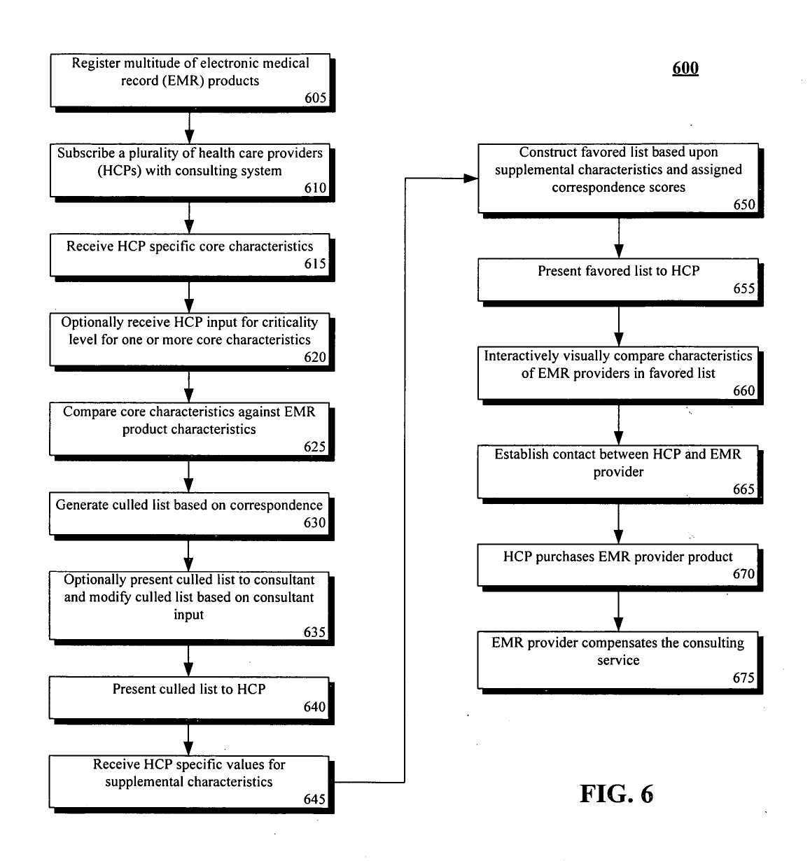

FIG. 6 is a flow chart illustrating a method 600 for matching EMR products to HCPs in accordance with an embodiment of the inventive arrangements disclosed herein. The method 600 can be performed in the context of a consultation system, such as the system 100.

Method 600 can begin in step 605, where a multitude of EMR products can be registered with a consulting service. The registration can be automated using a Web-based interface. Registration can involve the completion of a form detailing a multitude of standardized characteristic, established and defined by the consultation service.

In step 610, one or more HCPs can subscribe to the consulting service. The subscribing process can require each HCP to specify values for a number of core characteristics that the HCP requires. In step 615, the HCP specified core characteristics can be conveyed by the HCP to the consulting service. For example, the HCP can use a Web-based interface to provide the consulting service with input. In step 620, the HCP can optionally specify a criticality level for each core characteristic, which can be used to preferentially weigh selected core characteristics more than others.

In step 625, HCP specified core characteristics can be compared against EMR product characteristics. In step 630, a culled list of EMR products can be generated based upon a correspondence of core characteristics. For example, each EMR product can be assigned a correspondence score. This correspondence score can determine whether an EMR product is included in the culled list or not. In one embodiment, a correspondence threshold can be defined and only those EMR products having a correspondence score above the threshold will be included in the culled list.

In step 635, a human agent of the consulting service can be optionally presented with the culled list. The human agent can modify the list based upon personal knowledge concerning the EMR products and the HCP. For example, the human agent can add annotations to the culled list, can manually remove one or more EMR product from the culled list, can manually add one or more EMR product to the culled list, and perform similar operations.

In step 640, the modified culled list can be presented to the HCP. The HCP can then manually manipulate the list, adding and deleting selections based upon preferences and HCP knowledge. In step 645, the HCP can also specify additional, supplemental characteristics, which can be conveyed to the consulting service. In step 650, a favored list can be constructed based upon the current culled list and the supplemental characteristics. The favored list can, for example, contain the top five EMR products.

In step 655, the favored list can be presented to the HCP. In step 660, the HCP can visually compare characteristics of the EMR products in the favored list. For example, a spreadsheet or chart can present a grid of core and supplemental offerings of each of the EMR products. Individual values can be hyperlinked, the selection of which triggers the presentation of a detailed window for the selected offering.

In step 665, contact can be established between the HCP and EMR producer. Contact can be anonymous or interpersonal. For example, contact can consist of a series of email questions and responses to the EMR producer and/or the consulting service. Contact can also include a live communication or an arrangement for a demonstration.

In step 670, the contact can result in the HCP purchasing one or more product from the EMR producer. In step 675, the EMR producer can compensate the consulting service for facilitating the sale. The method 600 can be repeated any number of times resulting in different EMR product-HCP pairings.

It should be noted that method 600 is presented for illustrative purposes only and that the invention is not limited to the precise details specified within this illustrative embodiment. Instead, the steps presented within FIG. 6 can be modified by one of ordinary skill in a number of ways without departing from the scope and spirit of the method presented herein.

For example, the compensation specified in step 675 need not be a royalty paid by an EMR producer on a per-sale basis. Instead, revenue for operating the consulting service can be generated through a registration or subscription charge, through a government grant, through a trust established by health insurance companies, or any other source.

The present invention may be realized in hardware, software, or a combination of hardware and software. The present invention may be realized in a centralized fashion in one computer system or in a distributed fashion where different elements are spread across several interconnected computer systems. Any kind of computer system or other apparatus adapted for carrying out the methods described herein is suited. A typical combination of hardware and software may be a general purpose computer system with a computer program that, when being loaded and executed, controls the computer system such that it carries out the methods described herein.

The present invention also may be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

This invention may be embodied in other forms without departing from the spirit or essential attributes thereof. Accordingly, reference should be made to the following claims, rather than to the foregoing specification, as indicating the scope of the invention.

What is claimed is:

1. A system for searching for vendor products comprising:
a consultant data store populated with standardized product characteristics for a plurality of products supplied by different vendors;
a search engine configured to compare customer preferences against product characteristic contained in the consultant data store;
a client interface including an input section and a result section;
the input section configured for customers to enter customer preferences into a plurality of consultant established fields; and
a result section configured to present a plurality of vendor products based upon search engine results, wherein an indicator based upon a level of correspondence between customer preferences and product characteristics is displayed next to each vendor product.

2. The system of claim 1, wherein the result section further comprises a display element showing a correspondence level between each customer preference and each product characteristic.

3. The system of claim 2, wherein the display element is color coded in accordance with the correspondence level.

4. The system of claim 1, wherein the product characteristics in the consultant data store include a set of core characteristics and a set of supplemental characteristics, wherein consultant established fields corresponding to the core characteristics initially appear in the input section, and wherein after said plurality of vendor products are presented in the result section responsive to customer preferences entered into the initially appearing consultant established fields, the input section is altered to include consultant established fields corresponding to the supplemental characteristics.

5. The system of claim 1, wherein an initial list of vendor products based upon the entered customer preferences generates a consultant list of vendor products, wherein a consultant provides input responsive to receiving the consultant list, wherein the plurality of vendor products presented in the result section are different from the vendor products of the consultant list, said differences resulting from the consultant input.

6. The system of claim 1, wherein at least one of the plurality of vendor products presented in the result section include a consultant provided note related to that vendor product.

7. The system of claim 1, wherein said plurality of products are electronic medical record products, and wherein the client interface is a Web-based interface configured for health care providers.

8. A system for selecting an electronic medical record (EMR) solution comprising:
a consultant database including standardized characteristics for a plurality of EMR products;
a health care provider (HCP) interface configured to identify HCP specific values of the standardized characteristics;
a filtration engine configured to generate a culled list of EMR products, said culled list including a subset of EMR products having standardized characteristics corresponding to said HCP specific values, said filtration engine further configured to narrow the culled list to generate a favored list, wherein narrowing is based upon additional input provided by a party to whom the culled list was presented;
a presentation interface configured to present the favored list to a HCP; and
a contact mechanism configured to facilitate communications between the HCP and at least one EMR product selected from the favored list.

9. The system of claim 8, further comprising:
a registration engine for registering EMR products within the system, wherein only registered EMR products are included in the plurality of EMR products.

10. The system of claim 9, further comprising:
a compensation mechanism wherein registered EMR products agree to pay a consulting fee to a consulting entity associated with said system based.

11. The system of claim 8, wherein the HCP interface is a Web-based interface in which the presenting step occurs, wherein the filtration engine automatically generates the culled list based upon HCP interface input.

12. The system of claim 8, further comprising:
a consultant interface configured to present the culled list to a consultant, receive consultant input, and generate the favored list based in part upon the consultant input.

13. The system of claim 8, further comprising:
an EMR interface linked to the registration engine configured to register EMR products and to receive input to populate the standardized characteristics for each registered EMR product.

14. The system of claim 8, wherein the consultant interface and the EMR interface are Web-based interfaces.

15. A service for matching electronic medical record (EMR) providers to health care providers (HCPs) comprising the steps of:
registering a multitude of EMR products with said service, said registering step including receiving and validating EMR product specific values for a plurality of EMR standardized characteristics;
subscribing a plurality of HCPs with said service, said subscribing step including receiving HCP specific values for core characteristics, which are a subset of said standardized characteristics;
generating a culled list of EMR products for subscribed HCPs based on a correspondence between the EMR specific values and the HCP specific values for the core characteristics;
receiving HCP specific values for supplemental characteristics, which are a subset of said standardized characteristics;
constructing a favored list of EMR products based upon a correspondence between the EMR specific values and the HCP specific values for the supplemental characteristics; and
presenting said favored list to an HCP for which the favored list was constructed.

16. The service of claim 15, wherein the multitude of EMR products include at least fifty EMR products, wherein the core characteristics include at least ten characteristics, and wherein the supplemental characteristics include at least twenty characteristics.

17. The service of claim 15, wherein each registered EMR product is required to provide EMR specific values for standardized characteristics defined as core characteristics, and wherein each registered EMR product is permitted to optionally provide EMR specific values for standardized characteristics defined as supplemental characteristics.

18. The service of claim 15, further comprising the step of:
receiving at least one HCP defined criticality level for at least one core characteristic, wherein the critically level is used to weight the associated core characteristic when generating the culled list.

19. The service of claim 15, further comprising the step of:
providing a web-based EMR interface and a Web-based HCP interface,
wherein the registering step uses input provided via the EMR interface, and wherein the subscribing step uses input provided via the HCP interface.

20. The service of claim 19, further comprising the step of:
presenting the favored list within the HCP interface, wherein the presenting step visually compares EMR specific core values for each EMR product in the favored list.

21. The service of claim 15, further comprising the steps of:
receiving consultant input; and
utilizing said consultant input when constructing said favored list from said culled list.

22. The service of claim 21, further comprising the step of:
providing a Web-based consultant interface through which the culled list is presented to a consultant and through which consultant input is received.

23. A method for matching electronic medical record (EMR) providers to health care providers (HCPs) comprising the steps of:
registering a multitude of EMR products with a service, said registering step including receiving and validating EMR product specific values for a plurality of EMR standardized characteristics;
subscribing a plurality of HCPs with said service, said subscribing step including receiving HCP specific values for core characteristics, which are a subset of said standardized characteristics;
generating a culled list of EMR products for subscribed HCPs based on a correspondence between the EMR specific values and the HCP specific values for the core characteristics;
receiving HCP specific values for supplemental characteristics, which are a subset of said standardized characteristics;
constructing a favored list of EMR products based upon a correspondence between the EMR specific values and the HCP specific values for the supplemental characteristics; and
presenting said favored list to an HCP for which the favored list was constructed.

24. A machine-readable storage having stored thereon, a computer program having a plurality of code sections, said code sections executable by a machine for causing the machine to perform the steps of:
registering a multitude of EMR products with a service, said registering step including receiving and validating EMR product specific values for a plurality of EMR standardized characteristics;
subscribing a plurality of HCPs with said service, said subscribing step including receiving HCP specific values for core characteristics, which are a subset of said standardized characteristics;
generating a culled list of EMR products for subscribed HCPs based on a correspondence between the EMR specific values and the HCP specific values for the core characteristics;
receiving HCP specific values for supplemental characteristics, which are a subset of said standardized characteristics;
constructing a favored list of EMR products based upon a correspondence between the EMR specific values and the HCP specific values for the supplemental characteristics; and
presenting said favored list to an HCP for which the favored list was constructed.
